(19) United States
(12) Patent Application Publication
(10) Pub. No.: US 2020/0030560 A1
(43) Pub. Date: Jan. 30, 2020

(54) ENDOTRACHEAL INTUBATION DEVICE

(71) Applicants: Thomas JENKYN, London (CA); Stephen PARKES, West Lorne (CA)

(72) Inventors: Thomas JENKYN, London (CA); Stephen PARKES, West Lorne (CA)

(21) Appl. No.: 16/526,207

(22) Filed: Jul. 30, 2019

Related U.S. Application Data
(60) Provisional application No. 62/712,168, filed on Jul. 30, 2018.

(51) Int. Cl. A61M 16/04

(52) U.S. Cl. CPC A61M 16/0447; A61M 25/1002; A61M 16/0463

(57) ABSTRACT

An endotracheal intubation device has a flexible main tube having an outer end and a patient end for permitting passage of air from an environment outside a patient into a trachea of the patient when the patient is intubated with the device. An inflatable and deflatable balloon is attached to the main tube at a position closer to the patient end than the outer end of the main tube. The balloon is inflatable in the trachea to immobilize the main tube in the trachea. The balloon has a wall and the wall has a recess in a top surface thereof for collecting fluid secretions of the patient intubated with the device to prevent the fluid secretions from passing down the trachea into lungs when the balloon is deflated in the trachea. Preventing fluid from reaching the lungs when the device is removed reduces risk of ventilator associated pneumonia (VAP).

### CROSS-REFERENCE TO RELATED APPLICATIONS

This application claims the benefit of United States Provisional Patent Application U.S. Ser. No. 62/712,168 filed Jul. 30, 2018, the entire contents of which is herein incorporated by reference.

### FIELD

This application relates to medical devices, in particular to endotracheal intubation devices.

### BACKGROUND

An endotracheal intubation (ETT) device is a flexible tube that is placed in the mouth and down through the larynx and into the trachea of a patient, usually an unconscious patient, to ensure an airway for the patient. The flexible tube is a ‘main’ tube, which permits ventilation of the patient's lungs.

FIG. 1 and FIG. 2 illustrate an example of a prior art endotracheal intubation device 100 comprising a main tube 101, the main tube 101 having an outer (machine) end 102 and an inner (patient) end 103. The machine end 102 is fitted with a connector 104 that permits connection to a mechanical ventilator or hand air bag (not shown). The machine end 102 is the end from which the ventilation enters the main tube 101 and on into a patient 110. The endotracheal intubation device 100 also serves as a conduit through which drugs can be administered to the patient 110. The main tube 101 is held in place with a cuff balloon 105 that is integrally formed with the main tube 101 at a location near the patient end 103 of the main tube 101, which is the end of the main tube 101 furthest into a trachea 111 of the patient 110. The main tube 101 of the intubation device 100 is usually a flexible plastic tube with an internal diameter of between 2.5 mm to 5.5 mm for pediatric patients, and between 4 mm and 9 mm for adult patients.

The balloon 105 is deflated when the intubation device 100 is inserted into the trachea 111. Once the intubation device 100 is inserted into the trachea 111, the balloon 105 is inflated. The balloon 105 holds the intubation device 100 in place by pushing against vocal folds of the larynx 112 and walls of the trachea 111, which prevents removal of the intubation device 100 from the trachea 111 via tension from the machine end 102 of the main tube 101. The balloon 105 is deflated again to facilitate removal of the intubation device 100 from the trachea 111.

The balloon 105 is inflated via an inflation tube 106, which is a flexible plastic tube having a smaller diameter than the main tube 101. At least a portion of the inflation tube 106 runs parallel to the main tube 101. At some point along the main tube 101, the inflation tube 106 enters the main tube 101 and passes along the main tube 101 toward the patient end 103 of the intubation device 100 and into the balloon 105. An open end 107 of the inflation tube 106 is open to the atmosphere. The balloon 105 is inflated by attaching a syringe (not shown) full of atmospheric air and emptying the syringe into the inflation tube 106, thereby forcing air into the balloon 105 to inflate the balloon 105.

A valve 108 keeps the balloon 105 inflated; therefore, the syringe can be removed from the open end 107 of the inflation tube 106. To deflate the balloon 105, an empty syringe is fitted to the open end 107 of the inflation tube 106. The air from the balloon 105 is drawn into the empty syringe. As the syringe is drawn full, the air in the balloon 105 is drawn out and the balloon 105 deflates. The endotracheal intubation device 100 may then be removed past the vocal folds and out of a mouth 113 of the patient 110.

When the intubation device 100 is kept in place in the unconscious patient 110 for longer than several days, a buildup of fluid secretions 115 tends to collect at a top surface of the balloon 105, as seen in FIG. 2. The top surface of the balloon 105 is closer to the mouth 113. The fluid secretions 115 come from the lining of the trachea 111, the esophagus, the larynx 112, the mouth 113 and the sinuses. Gravity causes the fluid secretions 115 to travel down the trachea 111 because focal folds and epiglottis are held open by the intubation device 100.

The balloon 105 forms a seal against the interior wall of the trachea 111 and against the vocal folds. The fluid secretions 115 pool in a ring around the balloon 105 and against the interior wall of the trachea 111 because the balloon 105 curves downward from its attachment to the main tube 101 out to the wall of the trachea 111. If the volume of fluid secretions 115 is large, then the pooling will completely cover the superior (top) surface of the balloon 105 and fill the trachea 111 above the balloon 105 as seen in FIG. 2.

Difficulty arises when the intubation device 100 needs to be removed or repositioned. When the balloon 105 is deflated, the balloon 105 ceases to form a seal against the interior wall of the trachea 111. Therefore, the collected fluid secretions 115 are no longer held by the balloon 105 and the fluid secretions 115 travel via gravity down in to the lungs. The fluid secretions 115 usually contain bacteria; therefore, the passage of fluid secretions 115 into the lungs often causes a condition called Ventilator Associated Pneumonia or VAP. VAP is a serious condition in vulnerable patients.

There remains a need for an endotracheal intubation (ETT) device that is less prone to causing Ventilator Associated Pneumonia (VAP).

### SUMMARY

There is provided an endotracheal intubation device comprising: a flexible main tube having an outer end and a patient end for permitting passage of air from an environment outside a patient into a trachea of the patient when the patient is intubated with the device; and, an inflatable and deflatable balloon attached to the main tube at a position closer to the patient end than the outer end of the main tube, the balloon inflatable in the trachea to immobilize the main tube in the trachea when the patient is intubated with the device, the balloon comprising a wall and the wall having a recess in a top surface thereof for collecting fluid secretions of the patient intubated with the device to prevent the fluid secretions from passing down the trachea into lungs of the patient when the balloon is deflated in the trachea.

In some embodiments, the recess is concave. In some embodiments, the recess may comprise a pocket open to the trachea between the wall of the balloon and an outer surface of the main tube.

In some embodiments, balloon forms a cuff around the main tube whereby a patient end portion of the wall of the balloon is sealingly attached to an outer surface portion of the main tube. In some embodiments, the recess may comprise an annular volume between the wall of the balloon and the outer surface portion of the main tube open to the trachea above the sealing attachment of the wall of the balloon to the outer surface of the main tube.

In some embodiments, the recess is maintained by struts between the wall of the balloon and the outer surface of the main tube.

In some embodiments, the balloon may comprise a lumen created by the wall of the balloon. The wall of the balloon where the lumen is created may wrap around the main tube and may be connected to the main tube in an airtight manner around the main tube at a first place closer to the outer end of the main tube and at a second place closer to the patient end of the main tube.

In some embodiments, the balloon may be symmetrical around a centerline of the main tube.

In some embodiments, the balloon may comprise a flexible plastic material that allows the balloon to inflate to a configuration that blocks the trachea and deflate to a configuration that allows removal of the device from the trachea without loss of shape or material integrity of the balloon.

In some embodiments, the device may further comprise an inflating tube for inflating and deflating the balloon. The inflating tube may provide a fluid connection between an interior of the balloon and the environment outside the patient when the patient is intubated with the device.

In some embodiments, the device may further comprise a draining tube for draining fluid secretions from the recessed portion. The draining tube may provide a fluid connection between the recess and the environment outside the patient when the patient is intubated with the device.

In prior art endotracheal intubation devices, the shape of the top surface of the balloon is generally convex from the center at the main flexible tube outward to the inner tracheal wall. When the balloons of the prior art devices are deflated, secreted fluids that have collected on the top surface when the balloon was inflated will permit passage of the fluids to the lungs of the patient. Thus, prior art balloon configurations do not prevent the passage of the pooled fluids past the deflated balloon and into the lungs when the balloon is deflated.

The endotracheal intubation device of the present invention has a recess or pocket, preferably concave, in the top surface of the cuff balloon. The recess causes the secreted fluids from the patient to pool within the recess, rather than on a convex top surface of the balloon as is the case with prior art. The concave or recessed surface may form a cup on the top balloon surface within which the secreted fluids will pool and not be allowed to pass down into the lungs.

When the balloon in the present invention is deflated, the pooled fluids that are in the recess on the top surface of the balloon remain trapped in the recess. By trapping the pooled fluids in the recess, the fluids are unable to pass the deflated balloon and down the trachea of the patient and into the lungs. The endotracheal intubation device can be removed from the patient with the pooled fluids remaining trapped in the recess on the top surface of the balloon so that the fluids are removed from the patient along with the endotracheal intubation device. The device can then be re-placed in the trachea of the patient having had the pooled fluids cleared. The ability to clear secreted fluids before the fluids enter the lungs is particularly important for unconscious patients who lack the ability to voluntarily cough to remove such fluids.

Further, for the same reasons as indicated above, the prior art endotracheal intubation devices do not prevent the passage of the fluids past the deflated balloon into the lungs when the device is repositioned. In contrast, because the pooled fluids in the recess on the top surface of the balloon in the present endotracheal intubation device remain trapped in the recess, the balloon can be re-inflated after deflation and the pooled fluids remain trapped in the recess on the top surface of the balloon. This allows the endotracheal intubation device to be repositioned in the patient without the pooled fluids being freed to pass the balloon and into the lungs.

Furthermore, the pooled fluids trapped in the recess in the top surface of the balloon can be removed, for example by suction, from the recess via a small diameter draining tube without requiring the removal of the device from the patient's trachea. The pooled fluids trapped in the recess in the top surface of the balloon can be directly suctioned from the recess via a separate suctioning device without the removal of the device from the patient's trachea. The small diameter draining tube may run along the main tube from the outer end (also called the machine end) to the recess in the top surface of the balloon so that the fluids that have collected within the recess can be removed.

Further features will be described or will become apparent in the course of the following detailed description. It should be understood that each feature described herein may be utilized in any combination with any one or more of the other described features, and that each feature does not necessarily rely on the presence of another feature except where evident to one of skill in the art.

### BRIEF DESCRIPTION OF THE DRAWINGS

For clearer understanding, preferred embodiments will now be described in detail by way of example, with reference to the accompanying drawings, in which:

FIG. 1 depicts a prior art endotracheal intubation device showing a larger flexible main tube and a smaller inflation tube for inflating and deflating a balloon at a patient end of the main tube;

FIG. 2 depicts the endotracheal intubation device of FIG. 1 in place within the trachea of an unconscious patient with the balloon at the patient end inflated;

FIG. 3 depicts a schematic drawing of an endotracheal intubation (ETT) device of the present invention with an inflated cuff balloon;

FIG. 4A depicts a magnified view of a balloon section of the endotracheal intubation device of FIG. 3;

FIG. 4B depicts the balloon shown in FIG. 4A disassociated from the main tube and other tubes;

FIG. 5 depicts an oblique view of the balloon section of FIG. 4A, where the near field view is a top side of the balloon and main tube, the far field is the patient end of the main tube that is deepest in a trachea, and the view shows a cup formed by a top surface of the inflated balloon integral with the main tube;

FIG. 6 depicts a lateral view across the top surface of the inflated balloon in the balloon section of FIG. 5;

FIG. 7 depicts a top view of the top surface of the inflated balloon of FIG. 6; and,

FIG. 8 depicts a lateral view of the inflated balloon of FIG. 6.

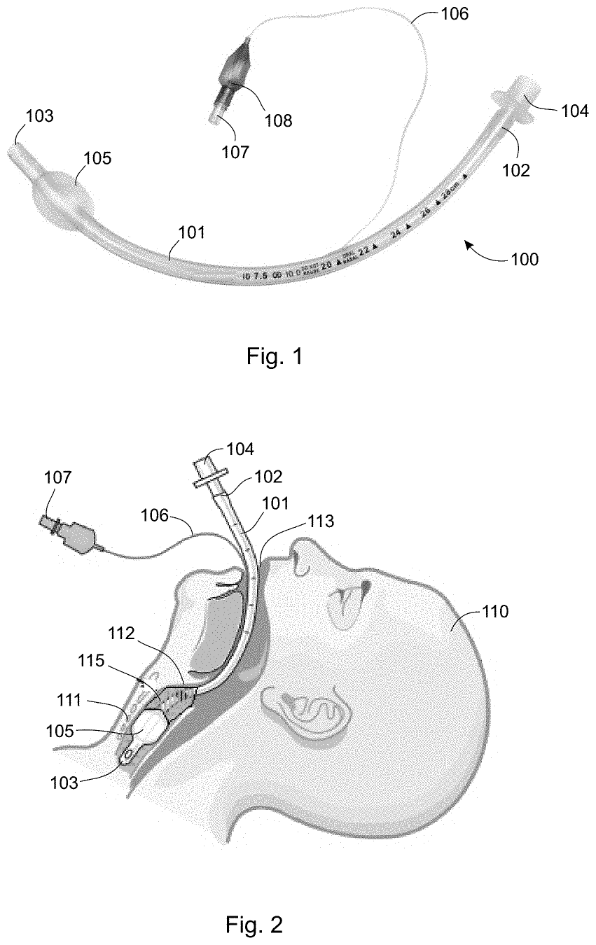

### DETAILED DESCRIPTION

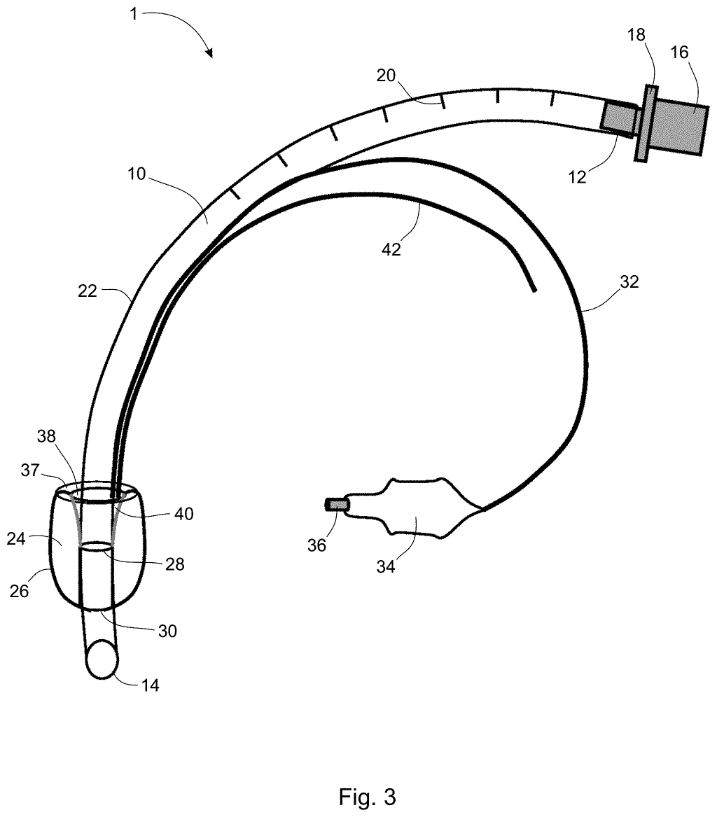

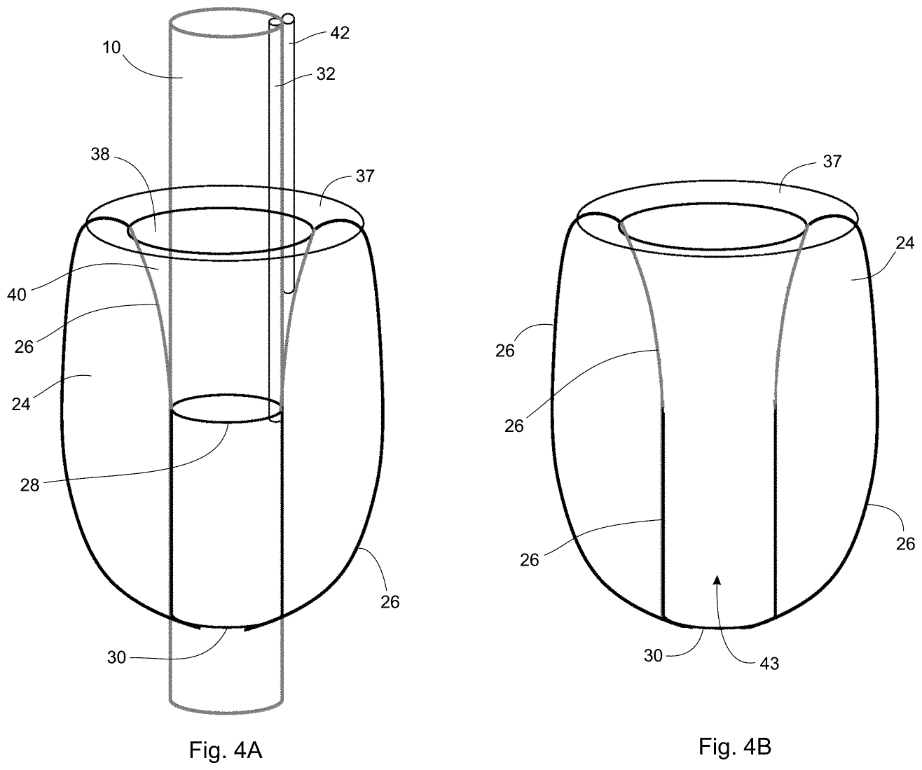

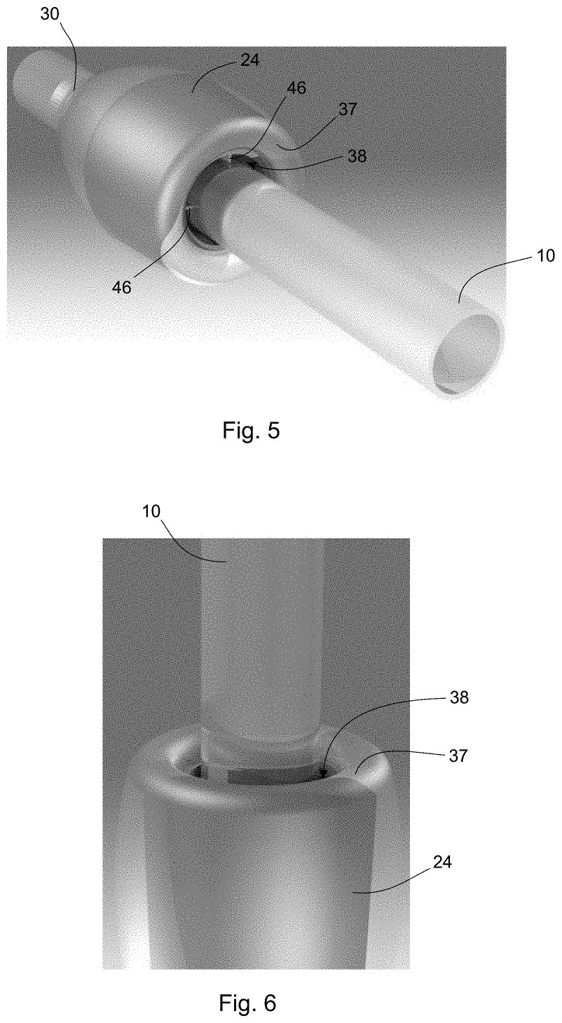

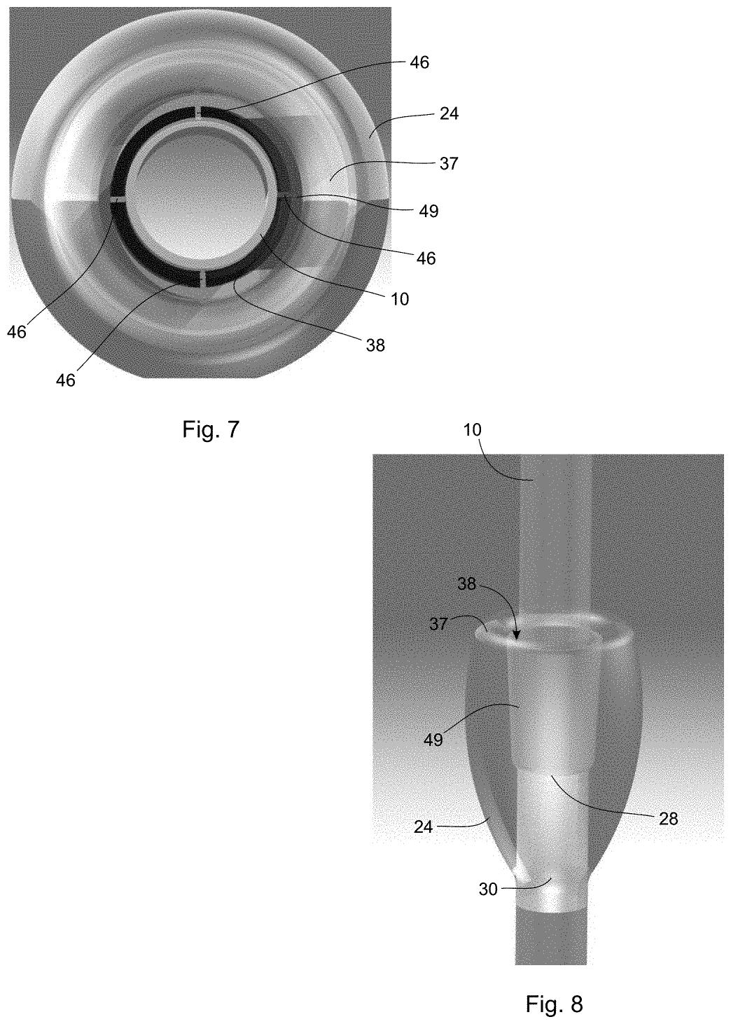

With reference to FIG. 3 to FIG. 8, a preferred embodiment of an endotracheal intubation device 1 of the present invention comprises a flexible plastic main tube 10. The main tube 10 has two ends 12, 14, where one end is an outer end 12 (also called a machine end), which is the end that remains exterior to a patient when the endotracheal intubation device 1 is positioned within a trachea of the patient, and where another end is a patient end 14, which is the end that is deepest within the patient when the endotracheal intubation device 1 is positioned within the trachea.

In a preferred embodiment the main tube 10 may comprise a medical grade polyvinyl chloride, which is a clear flexible plastic. In other embodiments the main tube may be composed of silicone rubber, latex rubber, polypropylene, polyethylene, polystyrene, polyethylene terephthalate or any other clear flexible plastic, semi-opaque flexible plastic or opaque flexible plastic. Preferably, the plastic of the main tube is safe for use in human patients according to appropriate regulatory agencies.

The main tube 10 preferably has an inner diameter in a range of from 2.5 mm to 5.5 mm for pediatric patients or 4 mm to 9 mm for adult patients. The main tube 10 may be manufactured in multiple sizes of inner diameter at discrete increments between the above-mentioned ranges of diameters. Wall thickness of main tube 10 may be between 0.65 mm and 1.2 mm. However, the wall thickness may be greater provided flexibility of the main tube 10 is maintained. The main tube 10 may have a length of between 8 cm and 18 cm for pediatric patients or 18 cm and 21 cm for adult patients. The main tube 10 may be manufactured in multiple tube lengths at discrete increments between the above-mentioned ranges of lengths.

The machine end 12 of the main tube 10 may be fitted with a connector 16 having a flange 18, which is used for connecting the main tube 10 to an airbag or mechanical ventilator as with a conventional endotracheal intubation device. The patient end 14 of the main tube 10 may be cut with a beveled end. In other embodiments, the patient end may be a butt end or a second hole may be provided in the wall of the main tube near the patient end (not shown) as with conventional endotracheal intubation devices.

The main tube 10 may comprise permanent visible markings 20 (only one labeled), which may be integral within the plastic of the main tube 10 to indicate the depth of the main tube 10 within the trachea. The main tube 10 may also have a radio-opaque wire 22 running along a length of the main tube 10, which is integral within the plastic of the main tube 10 or is adhered to an inside or outside of the main tube 10. The wire 22 is present so that the main tube 10 may be visualized with x-ray and computed tomography medical imaging.

The endotracheal intubation device 1 further comprises an inflatable and deflatable cuff balloon 24 attached to the main tube 10 closer to the patient end 14 of the main tube 10 than to the machine end 12. When the endotracheal intubation device 1 is positioned correctly within the trachea of the patient the balloon 24 is deep in relation to the vocal folds. The balloon 24, when inflated, holds the main tube 10 in place within the trachea by applying outward pressure on the inner wall of the trachea and upward pressure on the inferior surface of the vocal folds.

The balloon 24 comprises a lumen 43 created by a single flexible wall 26 of the balloon 24, the wall 26 attached to the main tube 10 at both ends of the lumen 43. The balloon wall 26 wraps around the main tube 10 and is connected in an airtight manner around the main tube 10 in two places: a first place 28 closer to the machine end 12 and a second place 30 closer to the patient end 14 of the main tube 10.

The balloon wall 26 is symmetrical around a centerline of the main tube 10. The balloon wall 26 preferably comprises a medical grade polyvinyl chloride, which is a clear flexible plastic. In other embodiments the balloon wall may be composed of silicone rubber, latex rubber, polypropylene, polyethylene, polystyrene, polyethylene terephthalate or any other clear flexible plastic, semi-opaque flexible plastic or opaque flexible plastic. The material of the balloon wall 26 is preferably flexible enough to allow the balloon 24 to inflate to the desired configuration and deflate to a configuration that allows for the endotracheal intubation device 1 to be removed from the trachea without loss of shape or material integrity. Also, the material of the balloon wall 26 is preferably flexible enough so that the balloon 24 can be inflated and deflated using a hand operated syringe without excessive resistance. Preferably, the plastic of the balloon wall 26 is safe for use in human patients according to appropriate regulatory agencies.

The balloon 24 may be inflated via an inflating tube 32 that runs along the main tube 10 on either the inner or outer surfaces of the main tube 10. The inflating tube 32 may leave the main tube 10 at some point along the length of the main tube 10 and is separate from the main tube 10 at the machine end 12 of the main tube 10. In another embodiment, the inflating tube may integral with the main tube along the entire length of he inflating tube. The balloon 24 may be inflated by forcing air into an out-of-patient end 34 of the inflating tube 32 with a syringe (not shown), which is temporarily attached to the inflating tube 32 at syringe connector 36. The balloon 24 may be deflated by forcing air out of the end 34 of the inflating tube 32 with a syringe that is temporarily attached at the syringe connector 36.

The balloon 24 comprises a concave recess 38 on a machine side (i.e. in a top surface 37) of the balloon 24. The recess 38 is created by the flexible wall 26 of the balloon 24 at the top surface 37 not attaching flush to main tube 10. Rather the recess 38 deepens to comprise a pocket 40 between the wall 26 of the balloon 24 and the main tube 10 bottoming where the wall 26 of the balloon 24 attaches at the first place 28 to the main tube 10. The balloon wall 26 at a patient end of the balloon 24 attaches flush with the main tube 10 of the endotracheal intubation device 1 at the second place 30 on the main tube 10.

The recess 38 in the top surface 37 of the balloon 24 may be accessed by a small diameter draining tube 42 that runs along the main tube 10 on either the inner or outer surfaces of the main tube 10. This draining tube 42 is used for draining fluids from the recess 38 of the balloon 24 without the need for removing the endotracheal intubation device 1 from the patient. The draining tube 42 may leave the main tube 10 at some point along the length of the main tube 10 and is separate from the main tube 10 at the machine end 12 of the main tube 10. In another embodiment, the draining tube may be integral with the main tube along its entire length. The recess 38 may be drained via the draining tube 42 by temporarily attaching a hand syringe (not shown) at an out-of-patient end 44 of the draining tube 42 and drawing the fluid into the syringe. In another embodiment, the recess 38 may be drained by temporarily attaching a suctioning device onto the end 44 of the draining tube 42 and suctioning the fluid out.

The recess 38 in the top surface 37 of the balloon 24 may be maintained by the stiffness of balloon wall 26 while it is inflated. However, the recess 38 may be further maintained by stiff struts 46 located between the balloon wall 26 and an outer surface of the main tube 10. The stiff struts may be formed in an annular support collar 49 disposed between the balloon wall 26 and the main tube 10. The number of struts may be one, or more than one. The struts 46 may comprise polyvinyl chloride, but in other embodiments, the struts can be composed of silicone rubber, latex rubber, polypropylene, polyethylene, polystyrene, polyethylene terephthalate or any other clear flexible plastic, semi-opaque flexible plastic or opaque flexible plastic. Preferably, the plastic of the struts 26 is safe for use in human patients according to appropriate regulatory agencies.

The endotracheal intubation device disclosed herein features a recess in the top surface of the cuff balloon. The cuff balloon serves to hold the device in position in the trachea of the patient when inflated. The device can be repositioned or removed from the trachea when the cuff balloon is deflated. The recess in the top surface of the cuff balloon is capable of trapping secreted fluids that would otherwise pass beyond the cuff balloon and into the patient's lungs, where the fluids could cause ventilator associated pneumonia (VAP). The recess is capable to retaining secreted fluids when the cuff balloon is deflated, and the device repositioned or removed from the trachea. The secreted fluids can be removed from the recess via a small diameter draining tube that communicates with the recess and is accessible from the patient's mouth. Also, the secreted fluids can be directly suctioned from the recess with the device in position in the trachea. By avoiding passage of secreted fluids into the lungs, the endotracheal intubation device improves the health and welfare of the intubated patient, especially when the patient is unconscious. Thus, use of the endotracheal intubation device of the present invention reduces risk of ventilator associated pneumonia (VAP) in patients who have been intubated.

The novel features will become apparent to those of skill in the art upon examination of the description. It should be understood, however, that the scope of the claims should not be limited by the embodiments but should be given the broadest interpretation consistent with the wording of the claims and the specification as a whole.

1. An endotracheal intubation device comprising:
a flexible main tube having an outer end and a patient end for permitting passage of air from an environment outside a patient into a trachea of the patient when the patient is intubated with the device; and,
an inflatable and deflatable balloon attached to the main tube at a position closer to the patient end than the outer end of the main tube, the balloon inflatable in the trachea to immobilize the main tube in the trachea when the patient is intubated with the device,
the balloon comprising a wall and the wall having a recess in a top surface thereof for collecting fluid secretions of the patient intubated with the device to prevent the fluid secretions from passing down the trachea into lungs of the patient when the balloon is deflated in the trachea.

2. The device of claim 1, wherein the recess is concave.

3. The device of claim 1, wherein the recess comprises a pocket open to the trachea between the wall of the balloon and an outer surface of the main tube.

4. The device of claim 1, wherein the recess forms a cup on the top surface of the balloon within which the fluid secretions pool to be prevented from passing down through the trachea into the lungs.

5. The device of claim 1, wherein the balloon forms a cuff around the main tube whereby a patient end portion of the wall of the balloon is sealingly attached to an outer surface portion of the main tube, and the recess comprises an annular volume between the wall of the balloon and the outer surface portion of the main tube open to the trachea above the sealing attachment of the wall of the balloon to the outer surface of the main tube.

6. The device of claim 2, wherein the recess is maintained by struts between the wall of the balloon and the outer surface of the main tube.

7. The device of claim 1, wherein the balloon comprises a lumen created by the wall of the balloon, whereby the wall of the balloon where the lumen is created wraps around the main tube and is connected to the main tube in an airtight manner around the main tube at a first place closer to the outer end of the main tube and at a second place closer to the patient end of the main tube.

8. The device of claim 1, wherein the balloon is symmetrical around a centerline of the main tube.

9. The device of claim 1, wherein the balloon comprises a flexible plastic material that allows the balloon to inflate to a configuration that blocks the trachea and deflate to a configuration that allows removal of the device from the trachea without loss of shape or material integrity of the balloon.

10. The device of claim 1, further comprising an inflating tube for inflating and deflating the balloon, the inflating tube providing fluid connection between an interior of the balloon and the environment outside the patient when the patient is intubated with the device.

11. The device of claim 1, further comprising a draining tube for draining fluid secretions from the recessed portion, the draining tube providing fluid connection between the recess and the environment outside the patient when the patient is intubated with the device.
